(19) United States
(12) Patent Application Publication
(10) Pub. No.: US 2025/0292878 A1
(43) Pub. Date: Sep. 18, 2025

(54) System for Medicinal Supply Monitoring and Management for Pooled Medicinal Product Across Studies

(71) Applicants: MSD International GmbH, Lucerne (CH); Merck Sharp & Dohme LLC, Rahway, NJ (US)

(72) Inventors: Rui Miguel Jeronimo Fernandes, Zug (CH); Martina Marauli, Lucerne (CH); Jeffrey M. England, Farmingdale, NJ (US); Lavina L. Pais, Morristown, NJ (US); Carol Ann Shultz Terkhorn, Lansdale, PA (US); Alexander Johannes Jozef Debets, Cham (CH)

(21) Appl. No.: 19/080,665

(22) Filed: Mar. 14, 2025

Related U.S. Application Data
(60) Provisional application No. 63/565,444, filed on Mar. 14, 2024.
(60) Provisional application No. 63/565,433, filed on Mar. 14, 2024.
(60) Provisional application No. 63/666,599, filed on Jul. 1, 2024.

(51) Int. Cl. G16H 10/20; G16H 30/40; G16H 40/67

(52) U.S. Cl. CPC G16H 10/20; G16H 30/40; G16H 40/67

(57) ABSTRACT

A medicinal supply management system manages distribution of medicinal supply. The system may implement a pooling algorithm for pooling studies together, across different countries, with different protocol labeling parameters and/or blinding parameters. The system may allocate unique identifiers for the pooled studies. The system may implement digital display labels on the medicinal product for up-to-date presentation of relevant information to healthcare providers administering the medicinal product. The system may further implement controlled packaging for maintaining proper storage of the medicinal product, e.g., during transport to the treatment site. The controlled packaging may further include sensors for monitoring the internal environment of the controlled packaging, to maintain proper storage of the medicinal product.

### CROSS-REFERENCE TO RELATED APPLICATIONS

The present application claims the benefit of and priority to U.S. Provisional Application No. 63/565,433 filed on Mar. 14, 2024, U.S. Provisional Application No. 63/565,444 filed on Mar. 14, 2024, and U.S. Provisional Application No. 63/666,599 filed on Jul. 1, 2024, all of which are incorporated by reference in their entirety.

### BACKGROUND

### 1. Technical Field

The subject matter described relates generally to a system for and management of medicinal products, e.g., in the context of investigation medicinal products (IMP) or commercial medicinal products.

### 2. Background Information

Clinical trial workflow is a multi-step process, each stage bringing its own complexities. Key steps include study design, participant enrollment, data collection, data analysis, and report generation. Regulatory compliance requires all clinical products to be comprehensively and accurately labeled. However, this process of labeling clinical products can create friction, especially with conventional physical labels.

The process of applying, maintaining, and reading physical labels is labor-intensive, slowing down the clinical trial workflow. More importantly, physical labels are prone to errors such as misprints, mismatches, and loss, each of which can compromise the integrity of the trial data. For example, if errors are identified in the physical labels or updates to the label are needed, a new set of physical labels need to be created, vetted, printed, quality approved according to regulations and attached to the entire batch of clinical product. This problem is further exacerbated if the clinical products have already been shipped to clinical sites or patient homes (e.g., in the context of at-home care), i.e., out of immediate access. Overall, these friction points in the labeling process create a burden on accuracy, efficiency, and cost-effectiveness, which can potentially jeopardize the success and validity of the clinical research trial itself.

Maintaining defined temperatures (e.g., ambient, cold or frozen) for certain types of medication used in clinical research trials can also be critical. Chemical composition and efficacy of these drugs can be significantly impacted when exposed to temperatures outside their recommended storage range. This practice, known as cold chain management, ensures that the potency, safety and accuracy of these drugs are not compromised at any point during the trial, subsequently ensuring the validity of the research results.

Vaccines, biological drugs, and many pharmaceutical compounds used in clinical trials need to also be appropriately allotted, uniquely identified, labeled, quality controlled, and distributed to clinical treatment sites. These trials may further require specialized equipment, e.g., laboratory equipment, ancillary equipment, adherence devices, etc. In some instances, the product (i.e., medicinal or equipment) can be common to a number of studies, further complicating the allotment decision. In many instances, some studies are conducted in many countries, where each may have its own disparate regulatory compliance requirements, e.g., around labeling.

Traditional systems have separate supply chain pipelines for allotting product for each study. This generally entails allocating the product to each study, at an early stage, without the ability to re-allocate product that is already assigned. For example, some regulatory compliance requirements do not permit a labeled product from being reassigned to another study. This leads to a lot of waste, with treatment sites having to dispose of unused or expired product that could have otherwise been repurposed for another study.

### SUMMARY

To address cold chain management, controlled packaging provides for a solution, with advanced active & passive cooling technologies and precise environment monitoring (e.g., temperature, vibration, light, humidity, etc.). The controlled packaging may further be connected (wired or wirelessly) to a computer networking environment that provides real-time monitoring data, e.g., useful for ongoing monitoring of storage conditions, alerting treatment-site researchers to remedy storage issues, providing insight into varying attributes of a clinical product, or indicating when a patient and/or clinical site may have opened the primary packaging.

To address labeling challenges, digital labels can provide a solution. Digital labels introduce several efficiencies to the clinical supply chain, benefiting key stakeholders including patients, clinicians, and health authorities. They provide flexibility and agility, improving both labeling and distribution operations, thus reducing the risk of delays or shortages and ensuring patient access. Digital labels can accommodate and present information in various languages as per patient preference, thus promoting diversity, equity, and inclusiveness in clinical trials. This adaptability eliminates the need for multilingual booklet labels or country-specific labeling, aligning with sustainability goals by reducing waste and fostering eco-friendly practices. The design of digital labels provides ample space to include more information as needed, and can display universally recognized icons to aid patient understanding

Accessibility and legibility of the digital labeling information are crucial considerations. Stakeholder feedback from varied users such as patients, pharmacists, and nurses across different settings like homes, clinical and sponsor sites is important to ensure this. For instance, a legibility study conducted on a prototype involving a small diverse patient population under both brightly and dimly lit conditions showed similar performance between digital labels and traditional paper labels. However, digital labels have the added benefit of being sturdier and environment resistant. This result underscores the potential of digital labeling technology as a safe and effective alternative to paper labels.

Moreover, digital labels on clinical products offer enhanced opportunities for reusability, thereby minimizing waste. Their digital nature allows for updates or changes to the label content without physical wastage or removal, opening up applications for relabeling or multi-phase trials without the need for new materials. Also, the labels allow unique capabilities, as having the potential to update information on labels in closed and sealed containers.

Further, a medicinal supply management system can solve the problems related to pooling of the product (i.e., medicinal supply or equipment) together and coordinating allocation, labeling, quality control, regulatory compliance, and distribution of the product across the disparate studies. Depending on local regulatory requirements, products may be labeled at a later point in the supply chain or a digital label may be updated as the assignment of the products particular studies is determined or updated.

### BRIEF DESCRIPTION OF THE DRAWINGS

FIG. 1 is a networked computing environment for managing clinical supply, according to one or more embodiments.

FIG. 2A is a first configuration of a controlled packaging with cooling, according to one or more embodiments.

FIG. 2B is an example arrangement of individually packaged medicinal products for shipping and distribution, according to one or more embodiments.

FIG. 3 is a second configuration of a controlled packaging with active cooling, according to one or more embodiments.

FIG. 4 is a third configuration of a controlled packaging with active cooling and a dispensing tray, according to one or more embodiments.

FIG. 5 is an illustrative overview of environmental conditions that can be monitored by controlled packaging, according to one or more embodiments.

FIG. 6 is an illustrative flowchart of encoding digital display label data on a digital display, according to one or more embodiments.

FIG. 7 illustrates pooling product across disparate studies by a medicinal supply management system, according to one or more embodiments.

FIG. 8 illustrates assignment of unique component identifiers to pooled product across studies by a medicinal supply management system, according to one or more embodiments.

FIG. 9 illustrates order fulfillment across the studies drawing from pooled product by a medicinal supply management system, according to one or more embodiments.

FIG. 10 illustrates labeling and protocol retrieval by an integration system in communication with the medicinal supply management system, according to one or more embodiments.

FIG. 11 illustrates a process flowchart describing a process of encoding label information on a digital display label of a medicinal product, according to one or more embodiments.

FIG. 12 illustrates a process flowchart describing a process of monitoring and managing medicinal product supply quality using controlled packaging, according to one or more embodiments.

FIG. 13 illustrates a process flowchart describing a process of management of pooled medicinal product across studies, according to one or more embodiments.

FIG. 14 is a block diagram of an example computing device, according to one or more embodiments.

### DETAILED DESCRIPTION

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### Medicinal Supply Management System Environment

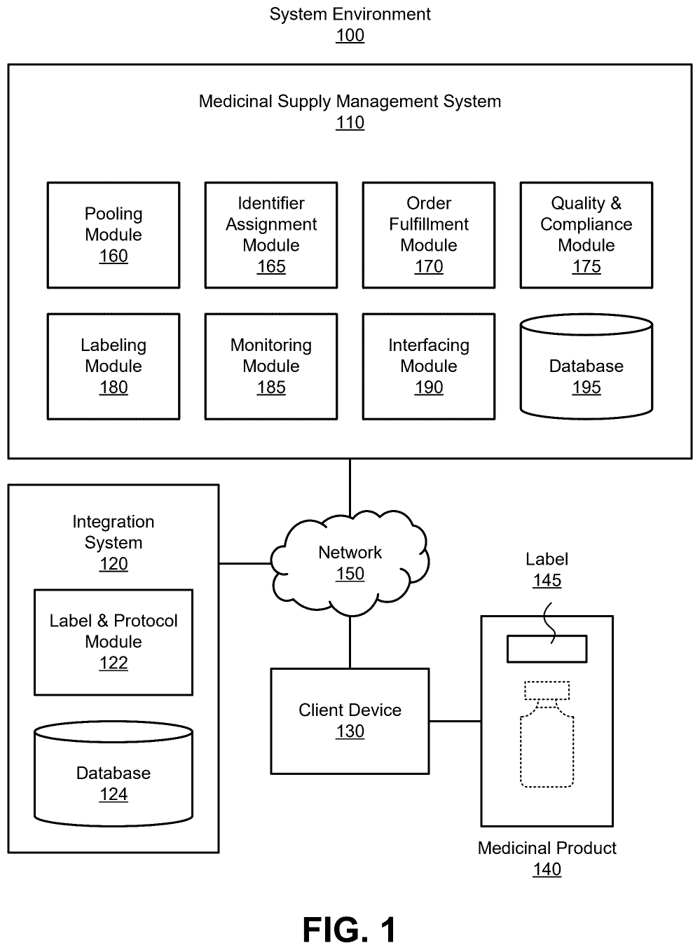

FIG. 1 is a block diagram of a networked computing environment suitable for monitoring and management of medicinal products, according to one or more embodiments. In the embodiment shown, the networked computing environment includes a medicinal supply management system 110, an integration system 120, a client device 130, and a network 150. In other embodiments, the networked computing environment includes different or additional elements. In addition, the functions may be distributed among the elements in a different manner than described. Furthermore, in another embodiment, the described functionality may be performed by a single computing device that is not connected to a network.

The medicinal supply management system 110 manages medicinal supply distributed to various treatment sites. For example, the medicinal supply management system may manage distribution of medicinal supply in clinical research trials. In another example, the medicinal supply management system may manage distribution of medicinal supply to other treatment sites, e.g., hospitals, medical clinics, etc. In yet another example, the medicinal supply management system may manage distribution of medicinal supply to patients for self-administration.

In managing the clinical research trials supply, the medicinal supply management system 110 may take part in any step in the clinical trial workflow, including: study design, participant enrollment, data collection, data analysis, and report generation. The medicinal supply management system 110 may include functionalities tailored to the unique demands of clinical research. The medicinal supply management system 110 may enable the setup of complex clinical studies, supports various study types and allows for the maintenance of clinically relevant parameters. The medicinal supply management system 110 may also provide proactive planning and forecasting, with functionalities to map out demand of medicinal product over the duration of the trial, carry out deterministic calculations, or simulate multiple planning scenarios based on enrollment figures. The medicinal supply management system 110 manages integration with devices and/or systems implemented at the treatment sites, e.g., using interfaces like the GS1 industry standard, Inventory Management systems, Building Management Systems, etc. The medicinal supply management system 110 further pools products together and manages the allocation, assignment of identifiers, labeling and protocol management, order fulfillment, quality and compliance checks of pooled product across the studies.

In one or more embodiments, the medicinal supply management system 110 comprises a pooling module 160, an identifier assignment module 165, an order fulfillment module 170, a quality and compliance module 175, a labeling module 180, and a database 195. In other embodiments, the medicinal supply management system 110 may include additional, fewer, or different components than those listed herein. In other embodiments, functionality of the components can be disparately divided among the system components.

The pooling module 160 pools products across studies together. The pooling module 160 implements an algorithm that inputs metrics related to disparate studies and outputs one or more pools that batch the studies together. The algorithm may further input other contextual data in determining the one or more pools, the other contextual data including country regulatory compliance requirements, blinding requirements, global standards, forecasted demand, etc. Pooling of the product provides the ability to coordinate management of the those across the studies. The algorithm may be a machine-learning algorithm configured to optimize the pooling. In some embodiments, the algorithm may be heuristic based on the regularly compliance requirements of countries, e.g., pool medicinal product across studies and countries with cross-applicable regulations. The pooling module 160 may store each pool of medicinal product in the database 195 in conjunction with various instructions or restrictions relating to the pool. For example, the instructions may include one or more workflows to enact when filling orders for the pool. In another example, the restrictions may include protocol labeling restrictions and blinding controls Furthermore, the pooling module 160 implements controls and validations that allow each clinical study to use pooled medicinal product from multiple different pools and features an enhanced algorithm for expiry-date-based planning of pooled medicinal product and automatic extension of master data to allow movements within the supply chain network.

The identifier assignment module 165 allocates pooled medicinal product across studies and assigns component identifiers (CIDs) to each medicinal product that are unique within the global portfolio of studies. The component identifier may be distinct from the digital display label unique identifier, e.g., in embodiments where the digital display label may be reused in packaging for different medicinal product 140. The identifier assignment module 165 may apply an algorithm that randomly assigns double permutated alphanumeric identifiers in a range to the product. Each component identifier for one unit of medicinal product is stored in the database 195. In some embodiments, each unit is associated with a manufacture serial number and the assigned component identifier. A medicinal product manufacturing system (not shown) manufactures medicinal product in batches that can be pooled across clinical studies. The manufacturing system informs the medicinal supply management system 110 of the batches manufactured. For example, one pooled medicinal product has a forecasted demand of ten thousand units for a clinical study and for a second clinical study has a forecasted demand of two thousand units. Accordingly, the medicinal supply management system 110 may proportionally allocated batches or units within each batch while executing the order fulfillment module 170.

The order fulfillment module 170 fulfills orders requesting medicinal product, e.g., received from treatment-site client devices 130. Each order request may specify an amount and the minimum expiry date of product being requested from the treatment site. The order fulfillment module 170 may operate in a first-in-first-out basis with order fulfillment. As orders are placed, the order fulfillment module 170 identifies what pooled study the order is associated with. The order fulfillment module 170 then identifies medicinal product allocated to the pool. If product is available, the order fulfillment module 170 may assign the available product to fulfilling the order. The product may be further assessed and evaluated for quality and regulatory compliance prior to distribution. Additional technical capabilities are created to restrict the usage of certain pooled medicinal product batches for a specific clinical study and/or country according to business rules (e.g., reserve some batches for countries that require longer expiry date shelf life), fully embedded in the batch determination logic. The product may also be labeled with the labeling module 180 before distribution.

The quality and compliance module 175 assess quality and compliance of product. The quality and compliance module 175 may perform appropriate checks of quality and compliance of product assigned to fill an order, e.g., by the order fulfillment module 170. The quality and compliance module 175 retrieves quality control checks and regulatory compliance requirements for the pool of studies associated with the order and the product. The quality and compliance module 175 performs the quality control checks for each product and may further certify the batch of products. The batch certification can boost efficiency in the certification process. The quality and compliance module 175 also assesses the regulatory compliance requirements. The regulatory compliance requirements may indicate labeling of the product, e.g., permissibility of just-in-time labeling, information required in labels, etc. The quality and compliance module 175 is able to retrieve information of the other modules on the Medicinal supply management system 110 and logically use it to enhance controls on pooled medicinal product, for example suggesting Qualified Person release certifications or study country releases based on the supply chain distribution network. In also incorporates logic to mass release a pooled medicinal product batch to several studies based on Pool master record.

The labeling module 180 manages labeling of each unit of medicinal product 140. In some instances, it is not possible to use pooled medicinal product as the country regulations require the protocol to be physically present on the label. In such instances, the labeling module 180 may generate an image for the label 145 for the medicinal product 140 to describe information relating to the medicinal product 140, e.g., component identifier, protocol, expiration date, or some combination thereof. The protocol may specify how a clinical trial will be conducted (the objective(s), design, methodology, statistical considerations and organization of a clinical trial,) and ensures the safety of the trial subjects and integrity of the data collected. The labeling module 180 provides the image of the label 145 for printing and affixing to the medicinal product 140. In some embodiments, the labeling module 180 splits up the label data into a plurality of pages that may be presented by the digital display (e.g., flipped through).

In other instances, the country regulations may allow pool with minimal labeling. In such instances, the labeling module 180 may generate an image for the label 145 for the medicinal product 140 that describes the component identifier, expiration date, storage conditions, or some combination thereof, but may omit the protocol. The labeling module 180 may provide the image of the label 145 for printing and affixing to the medicinal product 140. The protocol may be accessed by the client device 130 just-in-time for treatment of the patients at the treatment site. A new integration system 120 accessible by desktop or mobile devices is also available to retrieve protocol information at any time by scanning the barcodes belonging to Label 145.

In case a country regulation requires to have protocol information physically visible on the pooled medicinal product before shipment to a clinical site, the labeling module 180 may perform Just in Time (JIT) labeling (or e.g. Digital Display Label updates) triggered by the order fulfillment module 170. In performing JIT labeling, the labeling module 180 may perform line clearance, automation of product transfer to labeling stations, printing of JIT label, Digital Display Label updates or any type of label information presented to customers, label and component identifier reconciliation and review/approval procedures, or some combination thereof.

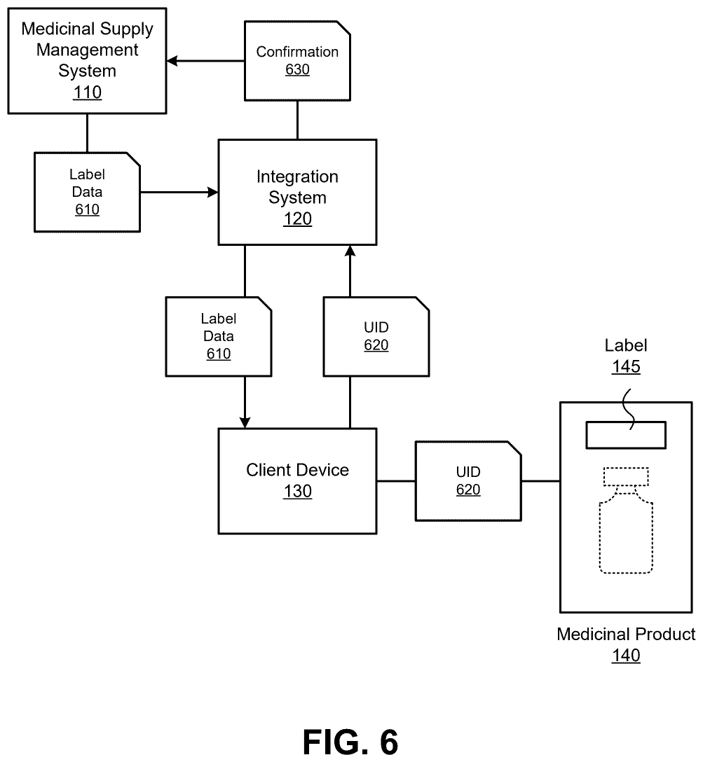

In other instances, the country regulations may allow for digital labeling. In such instances, the labeling module 180 may generate an image for the label 145. The image for the label 145 can be provided to the label 145, as a digital label, to present the relevant labeling information. In one or more embodiments, the labeling module 180 may also provide the relevant labeling information for a pool of studies (or for a particular protocol) to the integration system 120 to provide the image to the label 145, as the digital label. The digital label includes an electronic display that can display the relevant information. To update the label, the labeling module 180 may generate a new or updated image of the label (e.g., with updated protocol, updated storage conditions, etc.). The labeling module 180 provides the new or updated label to be presented by the label 145, as the digital label. Transmitting label information (i.e., label data) from the medicinal supply management system 110 to the label 145 may include leveraging the client device 130, an example of which is described in FIG. 6.

The monitoring module 185 receives monitoring data indicating status of the medicinal product 140. The monitoring module 185 may receive the monitoring data from the integration system 120 and/or the client device 130 in communication with a suite of one or more sensors coupled to the packaging for the medicinal product 140. The monitoring data may include data on any combination of environmental conditions, e.g., temperature, light, humidity, vibration, geographical position, etc. Each data stream can be tracked at an individual medicinal product level, or at a batch level. The monitoring module 185 may calculate a quality score for each unit of medicinal product based on the received monitoring data. Metrics for the quality score may be based on regulatory compliance requirements, medicinal product storage parameters, etc. The monitoring module 185 may store the monitored data in the database 195. The monitoring module 185 may leverage the monitored data in refining forecasting algorithms to forecast need across studies using pooled medicinal product. In some embodiments, the monitored data may also inform when a refill order of medicinal product is likely to happen. A notification may be generated and sent to the client device 130 to prompt the refill. In other embodiments, the medicinal supply management system 110 may track usage and anticipate needs across the studies using pooled medicinal product. The usage can also inform assignment of medicinal product in the order fulfillment process.

The monitoring module 185 may provide instructions relating to the safe storage or handling of medicinal product 140. For example, the monitoring module 185 may provide instructions including storage parameters for the medicinal product 140. The monitoring module 185 may provide such instructions to users and/or to the treatment-site client device 130 for controlling operation of the medicinal product 140's controlled packaging. For example, the controlled packaging may implement cooling technology which can be toggled according to the storage parameters.

In some embodiments, the monitoring module 185 may perform one or more actions based on the monitored data and/or the quality score. For example, the monitoring module 185 may prepare a report for a human operator to identify and/or remedy quality issues with the medicinal product. The report may indicate a potential issue negatively impacting the quality. In one or more embodiments with digital display labeling, the monitoring module 185 may also update a display label on the medicinal product with some indication reflecting the monitored data and/or the quality score. For example, if a medicinal product was exposed to higher than an acceptable temperature range, the labeling module 180 may generate an update to the digital display labeling on the product packaging to indicate the non-compliant storage conditions. In some embodiments, the monitoring module 185 may coordinate distribution of additional medicinal product based on the monitored data and/or the quality scores.

In some embodiments, the monitoring module 185 may generate instructions to modify operation of controlled packaging for the medicinal product 140. For example, the monitoring module 185 may determine a drop in temperature in the medicinal product 140. The monitoring module 185 may determine that the temperature is outside of a tolerance range. In response, the monitoring module 185 may generate instructions to the controlled packaging to increase active cooling. For example, the controlled packaging may include a

The interfacing module 190 generates an interface for communicating information to a user. The interfacing module 190 may generate a graphical user interface for presenting the information. The information may be presented as visual elements in the graphical user interface. The graphical user interface may further include visual elements for providing input, e.g., to the medicinal supply management system 110. The graphical user interface may be displayed on the client device 130. In some embodiments, the client device 130 may be operated by a user overseeing the distribution of the medicinal product. In such embodiments, the graphical user interface may present information on the medicinal product distribution process. For example, the graphical user interface may present information relating to manufacturing of medicinal product and related contextual information (e.g., manufacture date, manufacture quantity, quality assurance and control, etc.), labeling for the medicinal product (e.g., status of label review, outdated labels, etc.), medicinal product monitoring data (e.g., monitoring data describing environmental data of the medicinal product, storage conditions, etc.), medicinal product supply across treatment sites, other data relating to supply of medicinal product, etc.

In one or more embodiments, the analyses monitoring the environment of the medicinal product 140 may be performed on a computer affixed to the controlled packaging and in communication with the suite of sensors. In such embodiments, the computer may perform the various functionality of the monitoring module 185 in monitoring and maintaining appropriate storage parameters for the medicinal product 140.

In some embodiments, the interfacing module 190 may generate a report detailing the information relating to distribution of the medicinal product. The interfacing module 190 may transmit the report to the client device 130 for presentation to the user. In other embodiments, the interfacing module 190 may generate a notification detailing some information. For example, the interfacing module 190 may push out a notification based on data received by the medicinal supply management system 110. For example, if a batch of product is deemed contaminated, or otherwise compromised, the interfacing module 190 may push out a notification to client devices 130 at the one or more treatment sites indicating the compromised product of that batch. The notification may further indicate component identifiers of the specific units of medicinal product. The notification may include instructions prompting a user to discard the compromised product.

In one or more embodiments, the medicinal supply management system 110 may apply one or more machine-learning models in analyses related to the medicinal supply. For example, the medicinal supply management system 110 may apply a machine-learning model in the pooling of medicinal product across disparate studies. In another example the medicinal supply management system 110 may apply a machine-learning model to predict forecasted need of medicinal product at the treatment sites, over the timespan of the study. In another example, the medicinal supply management system 110 may apply a machine-learning model in assessment of quality of medicinal product over its lifespan.

Machine learning models are computer algorithms designed to learn patterns from data, empowering computer-based predictions. These models may be of diverse structures, e.g., linear regressions for continuous value prediction, or neural networks with layers and nodes that pass information to transform an input into an output. Example model types include linear and logistic regression, decision trees, support vector machines (SVMs), neural networks, and transformers. Linear and logistic regression handle continuous and binary outcomes, respectively. Decision trees use hierarchical structures for decision-making. SVMs excel in classification by defining optimal data separation boundaries. Neural networks, particularly deep learning architectures, process information through interconnected layers of nodes. Transformers, a type of neural network architecture, use an attention mechanism to differentially focus the transformer on different tokens in the input data. Transformers may be leveraged in Natural Language Processing (NLP).

The training of these models may employ various techniques. Supervised learning utilizes labeled data, whereas unsupervised learning discovers patterns in unlabeled data, such as clustering. Semi-supervised learning combines both approaches. Training may also include batching, which processes data in subsets, epoch training, which iterates through the dataset, fine-tuning, which adapts pre-trained models, retraining, which updates models with new data, and clustering, which groups similar data points. These methods collectively refine a model's predictive or classification accuracy.

The integration system 120 aids in management of label and protocol information between the medicinal product 140 and the medicinal supply management system 110. The integration system 120 may be one or more computing devices. In some embodiments, the integration system 120 may be implemented as cloud computing. The integration system 120 receives, from the medicinal supply management system 110, label, protocol, or other relevant information for medicinal product distributed to the pooled studies. The information may include images of labels and/or protocol information. The information may further include manufacturing information, storage information, expiration date, certifications, etc. The integration system 120 stores the information in the database 124. A label and protocol module 122 of the integration system 120 may receive a request to access label and/or protocol information for a medicinal product 140. The request may include the component identifier of the medicinal product 140. The label and protocol module 122 looks up the component identifier in the database 124 to find the relevant information associated with the medicinal product 140. The relevant information can be pushed back to the client device 130 in response to the request.

In one or more embodiments, the integration system 120 receives monitored data for sensors implemented in a controlled packaging for a medicinal product 140. The integration system 120 may provide the monitored data to the monitoring module 185 of the medicinal supply management system 110. The integration system 120 may also interact with any digital display labeling affixed to any used packaging of shipping container for the medicinal product, the medicinal product 140 and/or the controlled packaging. In such implementations, depots, storage locations or warehouses to store the product, the client device 130 or the end-user (e.g., patient) may provide label updates to the digital display labeling when having the appropriate privileges. In some embodiments, the client device 130 is implemented as part of the controlled packaging.

The client device 130 is a computing device with which a user may interact with the other elements of the networked computing environment (e.g., a terminal, laptop, tablet, smartphone, or any other suitable computing device).

In one or more embodiments, the client device 130 may be implemented at a distribution center for the medicinal product supply. The client device 130 may be used in encoding label data to digital display labels as the label 145 on the medicinal product 140. In other embodiments, the client device 130 aids the treatment site in managing the supply of medicinal product 140 and operation of the study. The client device 130 may further push out updates from the medicinal supply management system 110. For example, the client device 130 may provide a notification on an update to the protocol associated with the medicinal product 140. In another example, the client device 130 may provide a notification on a compliance issue related to the medicinal product 140 (e.g., in a recall situation). In case of pooled medicinal product, the medicinal supply management system 110 uses additional algorithms to determine the batch and corresponding component identifiers to be allocated to the specific study order coming from client device 130. The client device 130 may, upon receipt of the ordered medicinal product 140, provide a confirmation of receipt of the medicinal product 140 back to the medicinal supply management system 110.

In one or more embodiments, the client device 130 may be associated with a third-party treatment site including healthcare providers administering medicinal product to clinical trial participants. The client device 130 may provide orders to the medicinal supply management system 110. The medicinal supply management system 110 fulfills the orders and sends an order filled confirmation receipt to the client device 130.

In other embodiments, the client device 130 may be a personal computing device of a patient, e.g., in the context of direct-to-patient medicine. In such embodiments, the client device 130 may communicate with the digital display label to ascertain doses taken by the patient. For example, if the client device 130, based on data received from the digital display label, determines that the patient has missed a dose, the client device 130 may provide a notification indicating the missed dose. The notification may further prompt the patient to take the dose, in adherence with a dosing schedule. In other embodiments, the client device 130 may monitor supply of the medicinal product for the patient. If the supply drops below a threshold, the client device 130 may transmit a notification to the medicinal supply management system 110 indicating the low supply.

In some embodiments, the client device 130 can access protocol information for the medicinal product 140, e.g., received from the medicinal supply management system 110. The client device 130 may generate the request, indicating the component identifier of the medicinal product 140. The client device 130 may receive the component identifier as input from a user, e.g., the user types in the alphanumeric component identifier affixed to the medicinal product 140. In other embodiments, the label 145 may store a code (e.g., a QR code, a barcode, an RFID tag, a Bluetooth, NFC or other wireless chip, etc.) that can provide the component identifier to the integration system 120. The client device 130 scans the code of the label 145. In embodiments with the QR code or the barcode, the client device 130 may, via a camera assembly, capture an image of the QR code or the barcode to extract the component identifier. In embodiments with the RFID tag and/or the Bluetooth or other wireless chip, the integration system 120 may have a corresponding scanner to scan the Barcode/RFID tag and/or the Bluetooth or other wireless chip to extract the component identifier. In some embodiments, the client device 130 may be part of the controlled packaging. In other embodiments, the client device 130 may be co-located with the label 145 and the medicinal product 140.

The digital display label 145 affixed to the clinical product 140 includes an electronic display for displaying a label for the clinical product 140. The digital display label 145 may further include a battery, a processing unit, a wireless transmitter and receiver, or other electronic components. The digital display label 145 may include unique identifier registered and tracked by the monitoring module 185. For added security, the digital display label 145 may implement security and/or accessibility restrictions to protect against tampering of the digital display label 145 by unauthorized devices. The electronic display may be sized proportionally to the clinical product 140 packaging. The electronic display may implement electronic ink display technology to mimic the appearance of ink on paper, which can reduce glare and strain on eyes.

In one or more embodiments, the digital display label 145 can incorporate one or more sensors for sensing an environment of the clinical product 140. Example sensors include: a temperature sensor, a humidity sensor, a light sensor, a barometer, a geographical positioning receiver, an inertial measurement unit, an accelerometer, vibration and/or motion sensor, or a force meter, etc. Other types of sensors may be implemented. The digital display label 145 may provide the sensed data to the medicinal supply management system 110 or the integration system 120.

In one or more embodiments, a light sensor may be included in the suite of sensors. The light sensor may include a photodiode that converts incoming light into an electrical signal. The photodiode comprises a semiconductor material that generates current proportional to the intensity of incident light. Exposure to light may degrade the medicinal product, such that measuring the light level may inform quality of the medicinal product over its lifespan.

In one or more embodiments, a temperature sensor may be included in the suite of sensors. The temperature sensor measures a temperature by converting thermal energy into an electrical signal. The temperature sensor includes e.g. a thermocouple with an electrical resistance or voltage dependent on temperature variations. The temperature sensor may measure the temperature change with the change in electrical resistance or voltage.

In one or more embodiments, a humidity sensor may be included in the suite of sensors. A humidity sensor measures the amount of moisture in the air by detecting changes in electrical properties related to water vapor. The humidity sensor may incorporate a capacitive or resistive electrical element with its electrical characteristics altered based on humidity levels.

In one or more embodiments, a geographical positioning system receiver may be included in the suite of sensors. A geographical positioning system receiver determines its location by processing signals from a network of orbiting satellites. The geographical positioning system may include an antenna to capture satellite signals and a processor to calculate position based on signal timing and satellite positions, or connect to other devices for geolocation.

In one or more embodiments, an IMU or an accelerometer may be included in the suite of sensors. The IMU (Inertial Measurement Unit) or accelerometer measures motion and orientation by detecting changes in acceleration and rotational forces. The IMU or accelerometer typically includes micro-electromechanical systems (MEMS) sensors that respond to physical forces.

In one or more embodiments, a motion or vibration sensor may be included in the suite of sensors. The motion or vibration sensor detects physical movement or oscillatory motion by converting mechanical energy into an electrical signal. The motion or vibration sensor employs one or more piezoelectric elements, accelerometers, or infrared detectors that respond to changes in position or vibration.

In one or more embodiments, a dose counter may be included in the suite of sensors. The dose counter tracks doses administered with the medicinal product. The dose counter may implement a mechanical dose counter that uses a gear or lever to precisely control administration of a dose. As the gear or level releases a dose, the dose counter may correspondingly indicate the doses administered, or the doses remaining. The dose counter may implement an electronic switch, optical detector, or another type of sensor for electronic dose counting.

In one or more embodiments, controlled packaging may store and maintain appropriate environmental conditions for the medicinal product 140. The controlled packaging may implement advanced cooling technologies with a suite of sensors to monitor storage conditions of the medicinal product 140. In general, the controlled packaging may be sized to fit the medicinal product 140. The medicinal product 140 may be medicinal product (e.g., pharmaceutical formulations, vaccines, biosimilars, antibodies, analgesics, antiviral medication, etc.), medical devices, sterile equipment, etc.

The advanced cooling technologies may include active and/or passive cooling. In the example of passive cooling, the controlled packaging may incorporate insulation with high thermal resistivity to minimize the exchange of heat from the external environment into the internal environment. In the example of active cooling, the controlled packaging may utilize cooling devices to extract heat from the internal environment and/or to slow the transfer of heat from the external environment into the internal environment. In some embodiments, the controlled packaging may implement both active and passing cooling.

In some embodiments, the controlled packaging may further implement digital display labeling which can be connected to all the sensors. In other embodiments, the controlled packaging includes a computing device, e.g., the treatment-site client device 120. The sensors may monitor environmental conditions at varying intervals, e.g., every minute, 2 minutes, 3 minutes, 4 minutes, 5 minutes, 6 minutes, 7 minutes, 8 minutes, 9 minutes, or 10 minutes.

The network 150 provides the communication channels via which the other elements of the networked computing environment communicate. The network 150 can include any combination of local area and wide area networks, using wired or wireless communication systems. In one embodiment, the network 150 uses standard communications technologies and protocols. For example, the network 150 can include communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 150 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 150 may be represented using any suitable format, such as hypertext markup language (HTML) or extensible markup language (XML). In some embodiments, some or all of the communication links of the network 150 may be encrypted using any suitable technique or techniques.

### Controlled Packaging Configurations

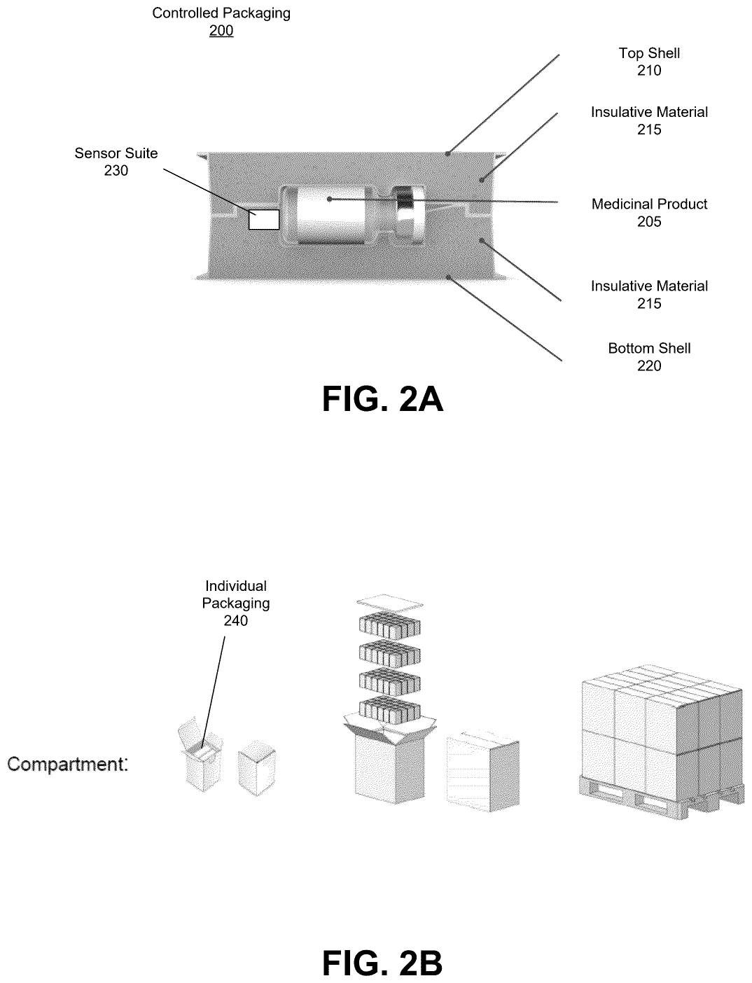

FIG. 2A is a first configuration of a controlled packaging 200 with active or passive cooling, according to one or more embodiments. As shown, the controlled packaging 200 includes a top shell 210 with insulative material 215, a bottom shell 220 with insulative material 215, and a sensor suite 230. In other embodiments, the controlled packaging 200 may include additional components, fewer components, or different components than those listed in FIG. 2A. For example, the controlled packaging 200 may include a digital display label.

The top shell 210 and the bottom shell 220 are complementary halves that securely store the medicinal product 205. In the example shown in FIG. 2A, the top shell 210 and the bottom shell both include respective insulative material to maintain a temperature range tolerance. In some embodiments, the top shell 210 and the bottom shell 220 may be coupled in a bivalve configuration, i.e., hinged along one side. The top shell 210 and the bottom shell 220 may be comprised of a stiff material, e.g., a hard thermoplastic. In other embodiments, the shells may be composed of other materials, e.g., metal, metallic alloys, hybrid materials, etc.

The insulative material 215 disposed in the top shell 210 and the bottom shell 220 is a material with high thermal resistivity (e.g., above 50 Kelvin-meter per watt (K·m/W)). In some embodiments, the insulative material 215 is a phase-change material that absorbs a large amount of heat to change phases, e.g., between solid to liquid (or gel-like). One example phase-change material is a salt hydrate, which is an inorganic compound that incorporates water molecules within the crystalline structure. In some embodiments, the insulative material 215 is at least partially dampening, capable of absorbing and dampening mechanical energy. Such dampening abilities help to mitigate fierce agitation of the medicinal product. The insulative material could also provide heat-exchange driven by an energy source, e.g., a battery.

The top shell 210 and the bottom shell 220 may be sized accordingly to fit the form factor of the medicinal product 205. In some embodiments, each unit of the medicinal product is in the form of a vial, a syringe, medical device, or an injector. Based on the form factor, the top shell 210 and the bottom shell 220 may be sized accordingly. In some embodiments, the size of the top shell 210 and the bottom shell 220 in relation to the medicinal product 205 is based in part on the storage parameters. For example, if the medicinal product 205 needs to be kept at 2-8° C. for a period of 48 hours, then the top shell 210 and the bottom shell 220 may be sized to fit a certain amount of insulative material 215, also possibly including an active (e.g., battery-driven) cooling unit.

The sensor suite 230 includes one or more sensors for monitoring environmental conditions of the medicinal product 205. The sensors may include, but are not limited to: a temperature sensor, a humidity sensor, a light sensor, a barometer, a geographical positioning receiver, an inertial measurement unit, an accelerometer, vibration/moving sensor, or a force meter, etc. The temperature sensor is configured to measure a temperature of the medicinal product 205, and/or the external environment temperature. The humidity sensor is configured to measure a humidity in the internal environment of the controlled packaging 200, i.e., wherein the medicinal product 205 is positioned during storage. The light sensor is configured to measure an amount of light incident on the internal environment. The geographical positioning receiver is configured to receive geographical positioning coordinates. The inertial measurement unit (IMU) is configured to measure rotational force and translational force on the medicinal product 205. The accelerometer is configured to measure translational force on the medicinal product 205. The force sensor is configured to measure an amount of mechanical force applied on the medicinal product 205. Another sensor can monitor whether an electronic tamper seal is broken (or opened). Other types of sensors may be implemented. The sensors measure the environmental data and provide the environmental data to another computing device (e.g., the treatment-site client device 120).

In one or more embodiments, the sensor suite 230 is positioned adjacent to the medicinal product 205 to capture environmental conditions or accessible digital data in proximity to the medicinal product 205. In other embodiments, the sensors may be variably disposed throughout the controlled packaging 200.

In one or more embodiments, the sensor suite 230 for each controlled packaging 200 may be in communication with other sensor suites for other controlled packaging. In such embodiments, the exchange of data may provide for improved monitoring. A monitoring system may aggregate the data to yield more confident evaluations of the storage conditions. For example, if one temperature sensor for one controlled packaging measures the present temperature to be outside the threshold range, but other temperature sensors measure the present temperature to be within the threshold range, the system may assess the uncorroborated reading as an outlier, e.g., perhaps due to a faulty sensor. In some embodiments, the system may leverage readings to cross-calibrate sensors, across controlled packaging.

FIG. 2B is an example arrangement of individually packaged medicinal products 240 for distribution, according to one or more embodiments. This figure illustrates three levels of packaging. In the first level, individually packaged medicinal products 240 (e.g., packaged in the controlled packaging 200) can be packed in pairs in one container. In the second level, the containers containing pairs of medicinal products can be rectilinearly packed into a batch container. In the third level, the batch containers can be rectilinearly packed onto a pallet for shipment. The pallet may comprise its own shell and insulative material, i.e., for passive cooling.

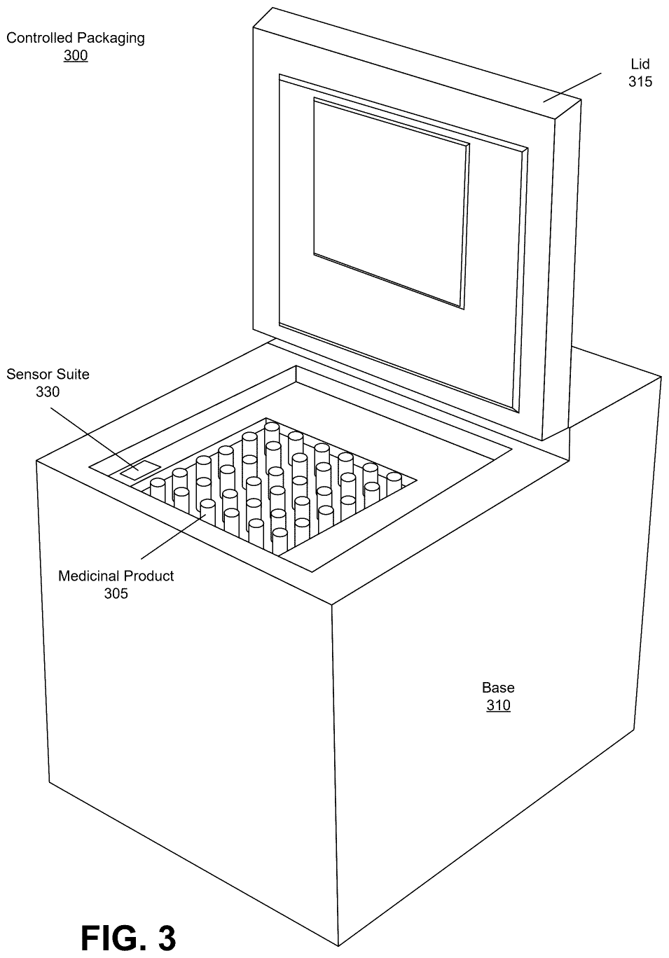

FIG. 3 is a second configuration of a controlled packaging 300 with active and/or passive cooling, according to one or more embodiments. The controlled packaging 300 implements active cooling to maintain an acceptable temperature range during storage and distribution of the medicinal product 305. In this configuration, the controlled packaging 300 includes a base 310, a lid 315, and a sensor suite 330 (an embodiment of the sensor suite 230). The controlled packaging 300 may further include one or more output devices for presenting information, e.g., a display for presenting visual information, a speaker system for presenting audio information. For example, a digital display label may be implemented in the controlled packaging 300.

The base 310 and the lid 315 together form a container to store the medicinal product 305. In the illustration shown, the active cooling technology is applied to the batch of medicinal products. The active cooling technology may operate on electricity, e.g., the controlled packaging 300 can plug into an electrical outlet to actively cool the medicinal product 305. The controlled packaging 300 may further include an electrical battery to store charge for operating the active cooling and/or passive cooling technology (e.g., cool-packs and gels) when the controlled packaging 300 is disconnected from the electricity supply, e.g., during transport of the container. In some embodiments, the container contains insulative material, thereby implementing both passive and active cooling in conjunction. In other embodiments, the individually package medicinal products described in the first configuration may be stored in the controlled packaging 300 with active cooling.

The sensor suite 330 includes one or more sensors for monitoring environmental conditions. The sensor suite 330 is an embodiment of the sensor suite 230. The monitored data may be provided to a computing device (e.g., to the treatment-site client device 120), which may in turn provide the data to a monitoring system (e.g., the integration module 102).

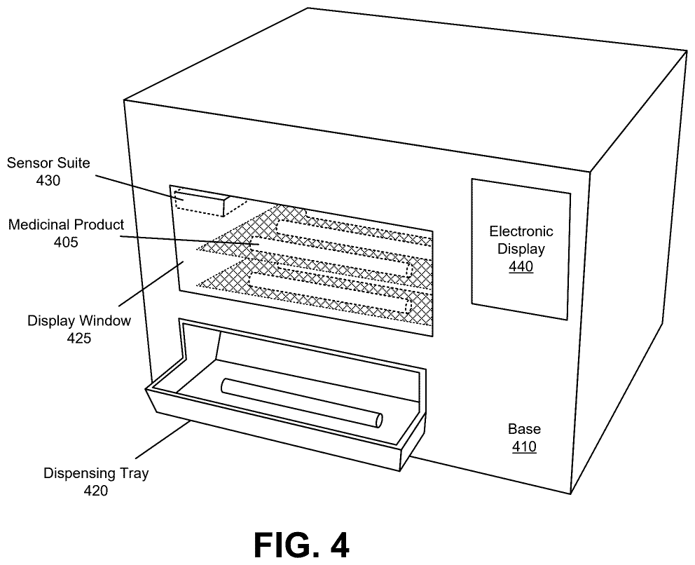

FIG. 4 is a third configuration of a controlled packaging with active cooling and a dispensing tray, according to one or more embodiments. The controlled packaging 400 implements active cooling to maintain an acceptable temperature range during storage and distribution of the medicinal product 405. In this configuration, the controlled packaging 400 includes a base 410, a dispensing tray 420, a display window 425, a sensor suite 430 (an embodiment of the sensor suite 230), and an electronic display 440.

The base 410 forms a container to store the medicinal product 405. In this configuration, the active cooling technology is applied to the batch of medicinal products. The active cooling technology may operate on electricity, e.g., the controlled packaging 400 can plug into an electrical outlet to actively cool the medicinal product 405. The controlled packaging 400 may further include an electrical battery to store charge for operating the active cooling when the controlled packaging 400 is disconnected from the electricity supply, e.g., during transport of the container. In some embodiments, the container contains insulative material, thereby implementing both passive and active cooling in conjunction. In other embodiments, the individually package medicinal products described in the first configuration may be stored in the controlled packaging 400 with active cooling.

In this third configuration, the controlled packaging 400 includes the dispensing tray 420 for dispensing of the medicinal product for use by a treatment-site researcher. The dispensing tray 420 may include an outward facing tray where the treatment-site researcher can obtain the dispensed medicinal product. The controlled packaging 400 may include a computing device to control the dispensing of the medicinal product. Such configuration limits exposure of the unused medicinal product from external environmental conditions. For example, the controlled packaging 400 may implement a secondary chamber that limits direct exposure with the external atmosphere, minimizing heat transfer into the container.

The display window 425 provides easy viewability by the treatment-site researcher, to assess inventory of the medicinal product. The display window 425 may be a double-paned with vacuum insulation, e.g., to mitigate heat transfer.

The sensor suite 430 includes one or more sensors for monitoring environmental conditions. The sensor suite 430 is an embodiment of the sensor suite 230. The monitored data may be provided to a computing device (e.g., to the treatment-site client device 120), which may in turn provide the data to a monitoring system (e.g., the integration module 102).

The electronic display 440 may provide real-time information to the treatment-site researcher. The electronic display 440 may present data relating to the environmental conditions measured by the sensor suite 430. For example, the electronic display 440 may provide a count of inventory, internal temperature of the controlled packaging 400, remaining shelf-life, etc. The electronic display 440 may further be a touch interface. In such embodiments, the treatment-site researcher (or a patient in the context of at-home care) can adjust the internal conditions, e.g., lowering temperature, etc. The treatment-site researcher may also use the touch interface to dispense the medicinal product. In some embodiments, the controlled packaging 400 may include a computing device that wirelessly communicates with other computing devices (e.g., the treatment-site client device 120).

The third configuration is particularly advantageous in improving patient adherence to the required dosing schedule. Such configuration can be implemented in clinical sites for streamlined efficiency in dispensing units of the medicinal product for use by the treatment-site researchers. The third configuration can also be implemented for at-home care. In such implementations, the controlled packaging's measured dispensing can aid to prevent overdosing and can aid in providing reminders to the patient if a dose has yet to be taken.

### Example Environmental Conditions

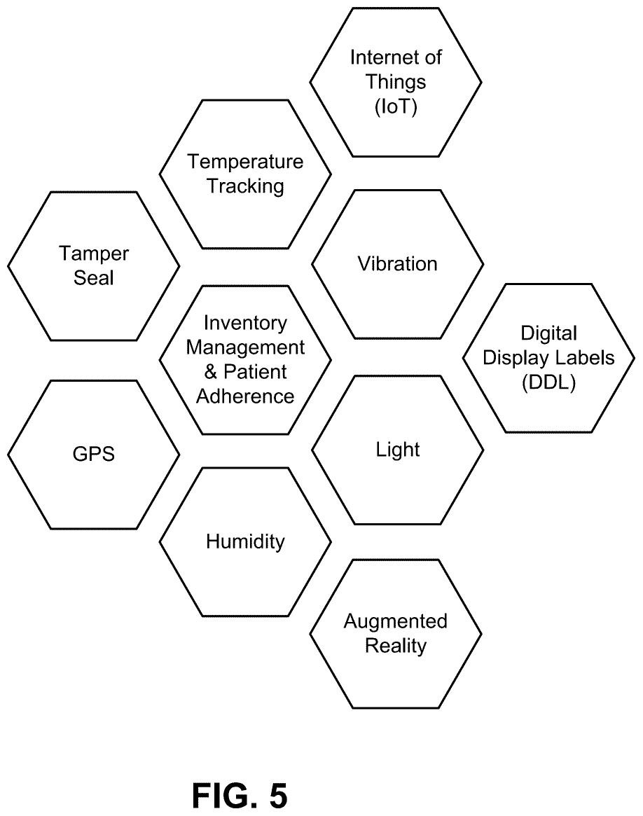

FIG. 5 is an illustrative flowchart of environmental conditions that can be monitored by controlled packaging, according to one or more embodiments. The monitoring and management of medicinal products in the context of clinical research trials may monitor a combination of the environmental conditions including: temperature tracking, vibration data, light exposure, humidity, global positioning system coordinates, breaking of a tamper seal. The tamper seal may be electronic, e.g., incorporating electronic circuitry, or magnetic contacts, to determine whether or not the controlled packaging has been opened, i.e., the tamper seal has been broken. The monitoring and management workflow may also incorporate connection to the Internet of Things (IoT), including smart phones, smart watches, smart glasses, personal computing devices, etc. The monitoring and management workflow may also work in conjunction with digital display labels (DDLs) affixed to the medicinal products. The monitoring and management workflow may also include one or more augmented reality implementations, e.g., for visualizing instructions to the treatment-site healthcare provider or direct to the patient.

### Example Workflows

FIGS. 6-10 provide example workflows of components in the system environment 100. The functionality described may be performed by other components than those described, or by other computing systems.

FIG. 6 illustrates encoding label data onto a digital display label as the label 145 of the medicinal product 140. The medicinal supply management system 110 provides the label data 610 to the integration system 120. The label data 610 may be previously approved, e.g., by a clinical trial overseer or a regulatory agency. The label data 610 may be stored in a database of the medicinal supply management system 110. The client device 130 connects to the label 145, e.g., via wireless communication technology. The client device 130 may identify the unique identifier (UID) 620 of the label 145. Based on the UID 620, the client device 130 may provide the UID 620 to the integration system 120. The integration system 120 may look up the appropriate label data 610 for the label 145, based on the UID 620. For example, the integration system 120 may maintain a table storing information related to each medicinal product allocated for a trial. The table may include information on medicinal product manufacture, protocols, formulations, packaging specifications, storage parameters, batch/lot numbers, CIDs, monitored data, etc. Upon identifying the appropriate label data 610, the integration system 120 may provide the label data 610 to the client device 130 to encode the label 145. In some embodiments, access to the label 145 may be limited to authorized client devices 130. A list of authorized client devices 130 may be stored by the integration system 120. In another embodiment, access may be limited to particular users, e.g., identified by user-specific login information. In some embodiments, transmissions between the various entities (e.g., between the client device 130 and the integration system 120, and/or between the medicinal supply management system 110 and the integration system 120) may be encrypted.

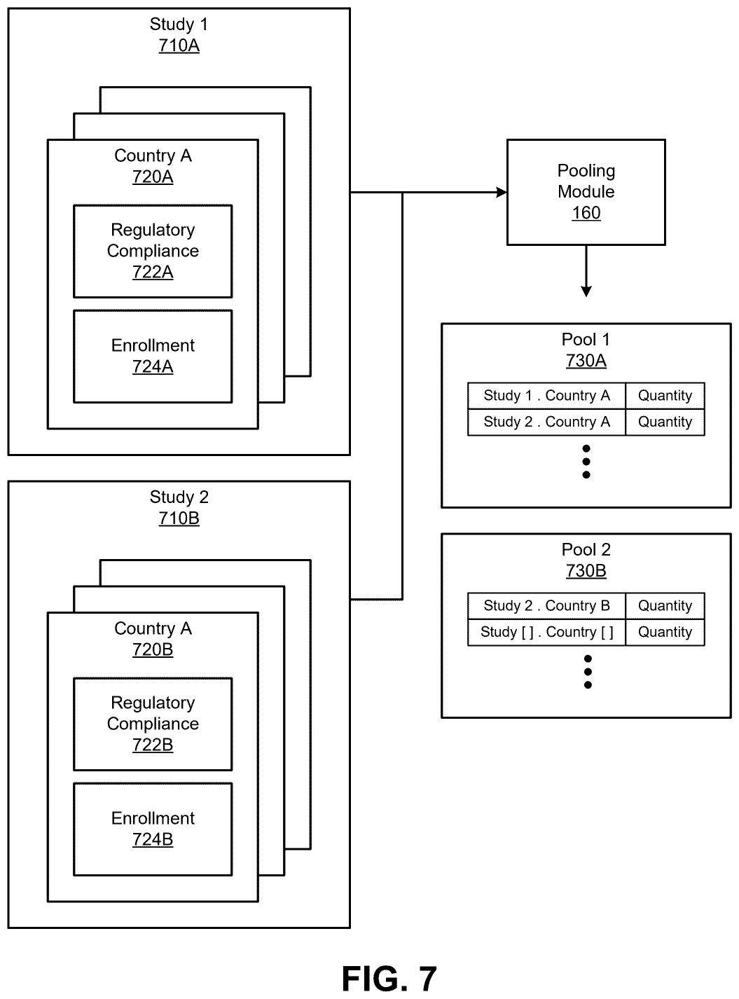

FIG. 7 illustrates pooling product across disparate studies by the medicinal supply management system 110, according to one or more embodiments. The pooling module 160 pools medicinal product across studies. For example, as illustrated, there are at least two concurrent studies leveraging the same medicinal product. Study 1 710A and Study 2 710B each include their own list of one or more countries with treatment sites enrolled in the study. For example, Study 1 710A includes enrollees in Country A 720A. As part of Study 1 710A in Country A 720A, Country A 720A has its own set of regulatory compliance 722A requirements and enrollment 724A, e.g., enrollment numbers, demographics, etc. As part of Study 2 710B in Country A 720B, Country A 720B shares the same list of regulatory compliance requirements 722B in Study 1 but may have differing enrollment 724B, e.g., enrollment numbers, demographics, etc. Such is the case with other countries under each study. Based on the characteristics of the studies, the pooling module 160 indicates which studies can use specific pooled medicinal products into a number of pools. Additional logic and validation are created to ensure that a pooled medicinal product can only be assigned in a study that is allowed based on pooling module 160. The pooling module 160 may apply a heuristic algorithm or a machine-learning algorithm to optimize the pooling. In the heuristic approach, the algorithm may form the minimum viable number of pools, then include studies into each pool based on study metrics, e.g., timing of study, protocol, country, labeling restrictions, etc. In the representative illustration, the pooling module 160 may generate at least two pools. Pool 1 730A pools at least the portion of Study 1 710A and Study 2 710B being performed in Country A. Pool 2 730B may include the portion of Study 2 710B being performed in Country B, and various other portions of studies. The pool lists can further include other relevant information, e.g., quantity of enrollees, etc.

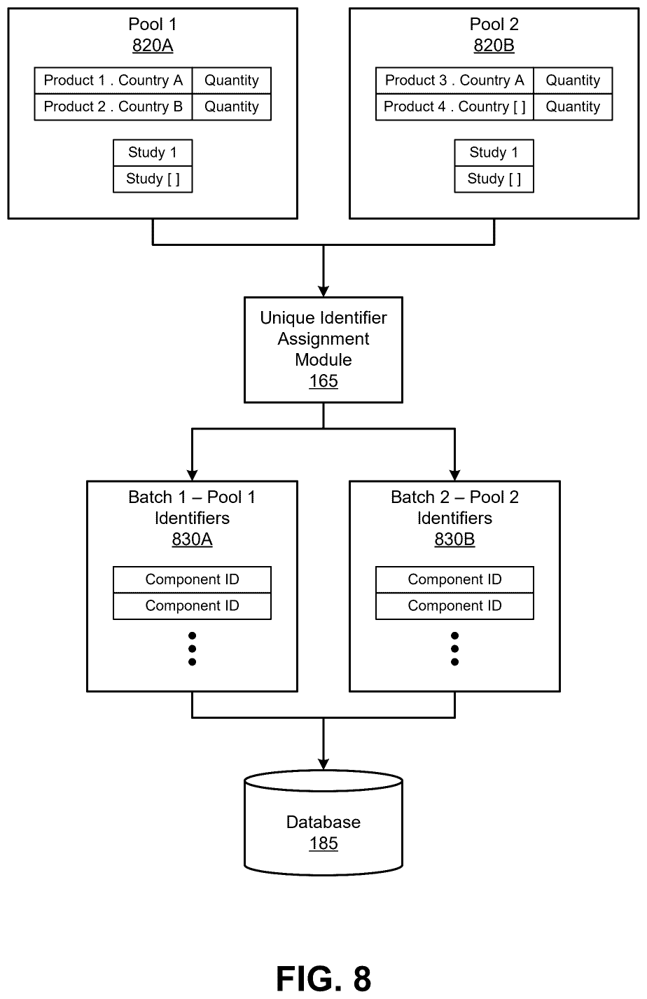

FIG. 8 illustrates assignment of unique component identifiers to pooled product across studies by the medicinal supply management system 110, according to one or more embodiments. The identifier assignment module 165 assigns unique component identifiers (CIDs or Component IDs) to the medicinal product to be manufactured. The identifier assignment module 165 creates batches of medicinal product for each pool. Within each pool, the identifier assignment module 165 assigns unique component identifiers to the medicinal product. The identifier assignment module 165 may store the unique component identifiers in the database 195. In the example shown, the unique identifier assignment module 165 may assign CIDs to medicinal product in Batch 1 allocated to Pool 1 820A. Likewise, the unique identifier assignment module 165 may assign CIDs to medicinal product in Batch 2 allocated to Pool 2 820B. The identifiers 830A for Batch 1 allocated to Pool 1 820A and the identifiers 830B for Batch 2 allocated to Pool 2 820B may be stored in the database 195.

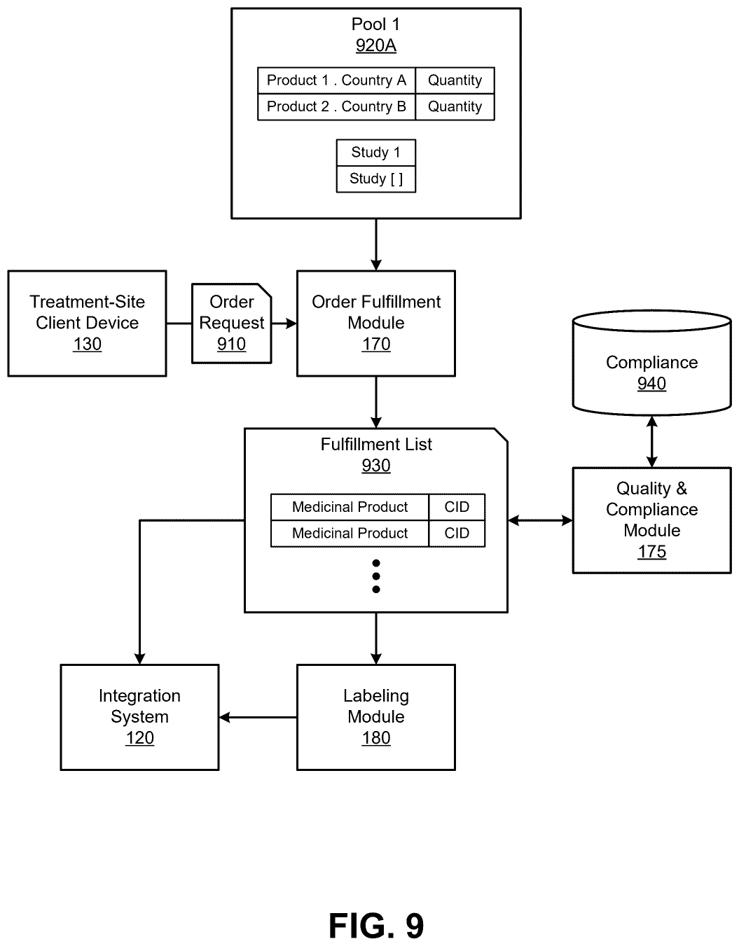

FIG. 9 illustrates order fulfillment across the studies drawing from pooled product by the medicinal supply management system 110, according to one or more embodiments. The order fulfillment module 170 receives an order request 910 from the client device 130. The order may indicate a quantity of medicinal product needed and other contextual information. For example, the medicinal supply management system 110 may provide an ordering interface that requests additional information, e.g., remaining supply, expiration date, urgency, anticipated need, etc. The order fulfillment module 170 receives the order request 910 and identifies the study and which pooled medicinal product the client device 130 is associated with. For example, the client device 130 may preregister itself with one of studies. Based on where the study is pooled, the order fulfillment module 170 may identify the appropriate Pool 1 920A via the ordered product. From Pool 1 920A, the order fulfillment module 170 may pull allocate medicinal product that is in the batches of product assigned to Pool 1 920A. Each medicinal product is provided a unique component identifier (CID). The order fulfillment module 170 collates medicinal product into a fulfillment list 930 to distribute or allocate to the treatment site that submitted the order request 910. The quality and compliance module 175 may, at this stage, perform the necessary checks to ensure the medicinal product in the fulfillment list 930 is ready for distribution. In some embodiments, the order fulfillment module 170 may perform just-in-time labeling by retrieving relevant label and/or protocol information to tag onto the fulfillment list 930, e.g., tagged to each CID. The label and/or protocol information may be stored in the integration system 120 for post-distribution access. The fulfillment list 930 dictates the allocation of the medicinal product to be distributed to the treatment site.

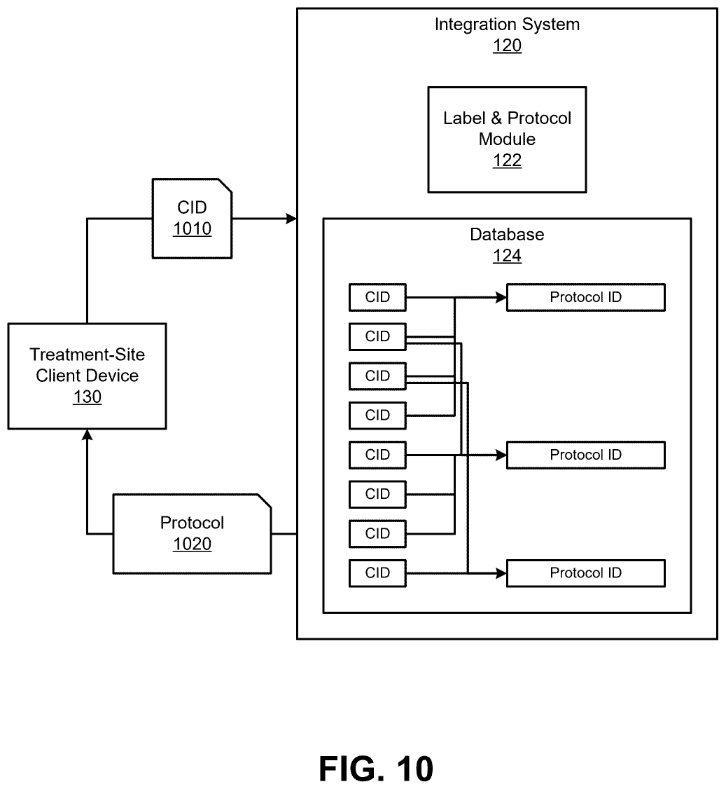

FIG. 10 illustrates labeling and protocol retrieval by the integration system 120 in communication with the medicinal supply management system 110, according to one or more embodiments. The integration system 120 may store the CID and the protocol information in the database 124. In some embodiments, each CID for a pool may be linked to one protocol ID, as determined when the medicinal product is allocated to a certain study and treatment site. In other embodiments, one medicinal product unit may be linked to multiple protocols, e.g., allowing the treatment site clinician to use the same unit in providing treatment to different studies in the pool. A user provides a CID for a medicinal product to the input screen of integration system 120 via barcode scanning. The label and protocol module 122 searches the database 124, for the matching CID. The label and protocol module 122 may pull the protocol ID linked to the CID and/or any other relevant information and provide that back to the display available on integration system 120 (mobile or desktop application). In some embodiments, the client device 130 may further provide an identifier related to a participant in one of the studies. The label and protocol module 122 may identify which protocol ID to pull from the database 124 based on the study identified for the participant.

### EXAMPLE METHODS

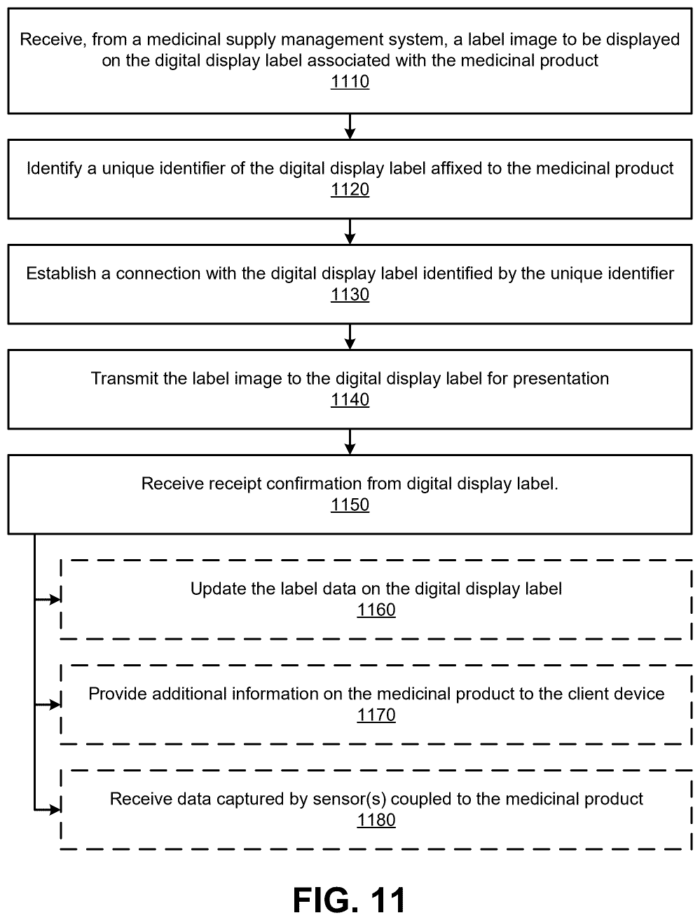

FIG. 11 illustrates a process flowchart describing a process of encoding label information on a digital display label of a medicinal product, according to one or more embodiments. The process may be performed by an integration system, e.g., in conjunction with a client device establishing connection to the digital display label for a medicinal product. In other embodiments, the process may include additional, fewer, or different steps than those listed herein.

The system receives 1110, from a medicinal supply management system, a label image to be displayed on the digital display label associated with the medicinal product. The label presents information on a formulation of the medicinal product, a protocol for administration of the medicinal product, information relating to storage parameters, expiry date, information on manufacture, etc.

The system identifies 1120 a unique identifier of the digital display label affixed to the medicinal product. To identify the unique identifier, the client device may scan an electronic signature of the digital display label indicating the unique identifier of the digital display label. The client device may scan the electronic signature by receiving the electronic signature with e.g. Bluetooth technology, radio frequency identification (RFID) technology, Zigbee, or near-field communication (NFC) technology. In other embodiments, to identify the unique identifier, the client device can capture an image of a QR code or a barcode. The system identifies the unique identifier of the digital display label through image analysis of the image of the QR code or the barcode.

The system establishes 1130 a connection with the digital display label identified by the unique identifier. The system may establish the connection via the client device, leveraging the client device to transmit the label image to the digital display label. The client device may further

The system transmits 1140 the label image to the digital display label for presentation.

The system receives 1150 a receipt confirmation from the digital display label. The receipt confirmation confirms the digital display label's receipt of the label image. The system may store confirmations for compliance auditing.

In some embodiments, the system may update 1160 the label data on the digital display label. The system receives, from the medicinal supply management system, an updated version of the label image. The system transmits, to the client device, a notification indicating presence of the updated version of the label image. The client device can re-establish connection to the digital display label and transmit the updated version to the digital display label for presentation.

In some embodiments, the system may provide 1170 additional information on the medicinal product to the client device. The system may receive, via the connection to the digital display label, a request to view a protocol associated with the medicinal product. The system identifies, e.g., in a table or a database, the protocol associated with the unique identifier of the digital display label affixed to the medicinal product. The system transmits the protocol to the digital display label for presentation.

In some embodiments, the system may further receive 1180, via the connection to the digital display label, data captured by one or more sensors coupled to the medicinal product; and storing the data captured by the one or more sensors in conjunction with the unique identifier for the digital display label.

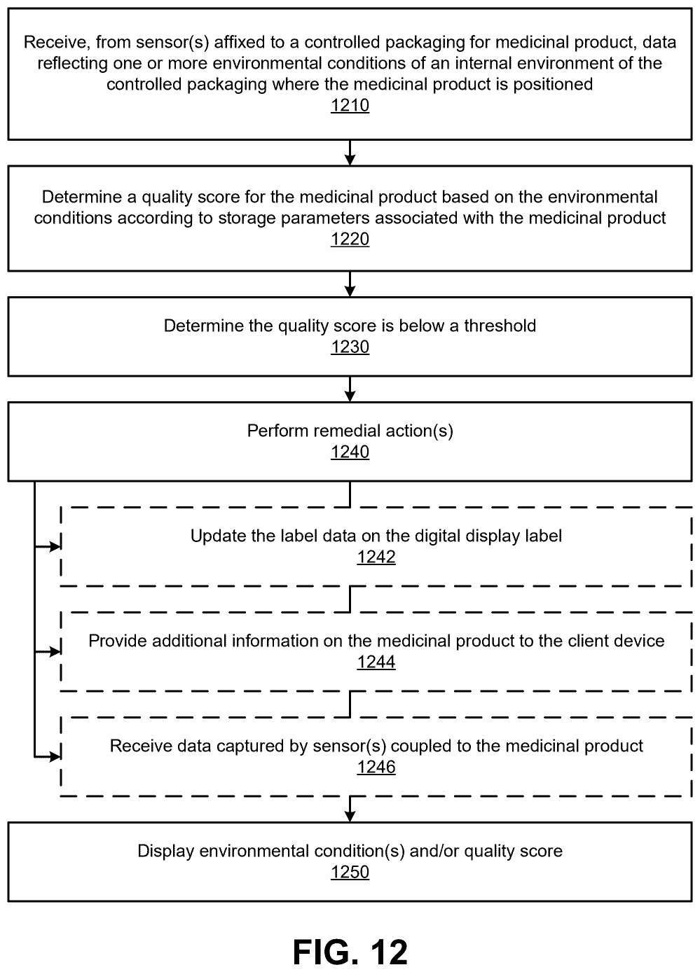

FIG. 12 illustrates a process flowchart describing a process of monitoring and managing medicinal product supply quality using controlled packaging, according to one or more embodiments. The process may be performed by an integration system, e.g., in conjunction with controlled packaging for storing of the medicinal product. In other embodiments, a client device or another system may perform the process. In other embodiments, the process may include additional, fewer, or different steps than those listed herein.

The system receives 1210, from one or more sensors affixed to a controlled packaging for medicinal product, data reflecting one or more environmental conditions of an internal environment of the controlled packaging where the medicinal product is positioned. The data may include temperature data, humidity data, light data, IMU data, accelerometer data, motion data, vibration data, GPS data, etc.

The system determines 1220 a quality score for the medicinal product based on the environmental conditions according to storage parameters associated with the medicinal product. The controlled packaging may include active cooling and/or passive cooling. The passive cooling mechanism may be comprised of two shells filled with a phase-change material with high thermal resistivity. In determining the quality score, the system may initially determine a score for each environmental condition based on the storage parameters associated with the medicinal product, then determine the quality score as a weighted sum of the scores for the environmental conditions.

The system determines 1230 that the quality score is below a threshold.

The system performs 1240 one or more remedial actions. As one example remedial action, the system transmits 1242 a report to an operator. The report may detail the compromised quality. The report may include instructions or recommendations for remediating storage. The report may further detail the quality score for the medicinal product. As another example remedial action, the system transmits 1244 instructions to the controlled packaging to adjust operation. The system may control, for example, an active cooling mechanism of the controlled packaging. As another example, the system may transmit 1246 a notification to a medicinal supply management system indicating a refill need for the medicinal product.

The system may display 1250 the environmental conditions and/or the quality score on a digital display label affixed to an external surface of the controlled packaging.

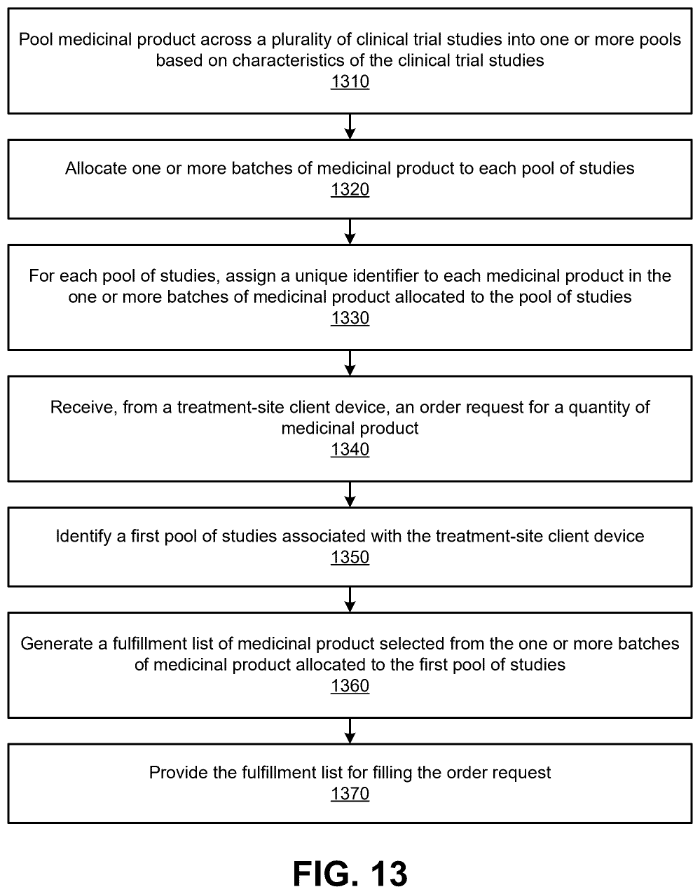

FIG. 13 illustrates a process flowchart describing a process of management of pooled medicinal product across studies, according to one or more embodiments. The process may be performed by a medicinal supply management system. In other embodiments, a client device or another system may perform the process. In other embodiments, the process may include additional, fewer, or different steps than those listed herein.

The system pools 1310 medicinal product across a plurality of clinical trial studies into one or more pools based on characteristics of the clinical trial studies. Pooling may entail: identifying, for each clinical trial study, a set of one or more countries for conducting the clinical trial study; identifying, for each country, protocol labeling parameters; and pooling the medicinal product based on overlapping protocol labeling parameters between countries. Pooling may entail: identifying, for each clinical trial study, blinding parameters; and pooling the medicinal product based on the blinding parameters for the clinical trial studies.

The system allocates 1320 one or more batches of medicinal product to each pool of studies. The system may apply a model (e.g., a machine-learning model) to forecast demand for each pool of studies based on the clinical trial studies in the pool of studies. With the forecasted demand, the system may allocate the batches of medicinal product across the pools of studies.

For each pool of studies, the system assigns 1330 a unique identifier to each medicinal product in the one or more batches of medicinal product allocated to the pool of studies. The system may perform random assignment, e.g., random assignment of double permutated alphanumeric identifiers

The system receives 1340, from a treatment-site client device, an order request for a quantity of medicinal product.

The system identifies 1350 a first pool of studies associated with the treatment-site client device.

The system generates 1360 a fulfillment list of medicinal product selected from the one or more batches of medicinal product allocated to the first pool of studies. The system may apply another model (e.g., a machine-learning model) to forecast demand for a treatment site associated with the treatment-site client device based on participants enrolled at the treatment site. The system may then generate the fulfillment list of medicinal product to allocate an amount of medicinal product based on the forecasted demand for the treatment site.

The system provides 1370 the fulfillment list for filling the order request. The system may further store the unique identifiers for the medicinal product for each pool of studies in a database associated with one or more protocols for the clinical trial studies in the pool of studies. Responsive to a request identifying a participant and the unique identifier for the medicinal product, the system may identify the appropriate protocol to transmit to the treatment-site client, the appropriate protocol associated with the study in which the participant is enrolled.

In some embodiments, the system, prior to shipping the order, may encode a label image for inclusion on the medicinal product allocated to the first pool of studies. The system may encode the label image for presentation on digital display labels affixed to the medicinal product allocated to the first pool of studies. In some embodiments, the label image is selected based on the country's protocol labeling parameters, blinding parameters, etc.

### Computing System Architecture

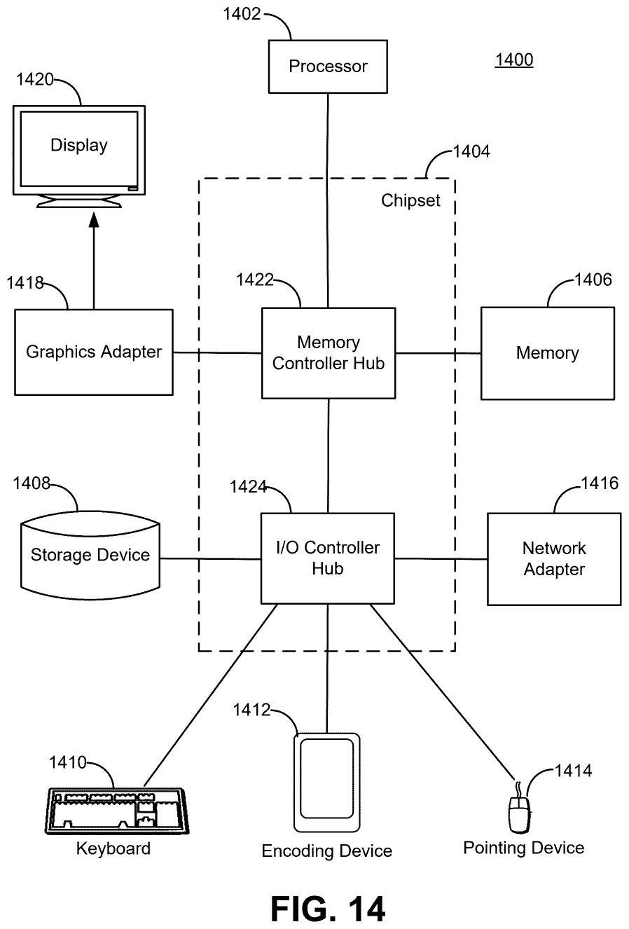

FIG. 14 is a block diagram of an example computer 1400 suitable for use in the networked computing environment. The example computer 1400 includes at least one processor 1402 coupled to a chipset 1404. The chipset 1404 includes a memory controller hub 1420 and an input/output (I/O) controller hub 1422. A memory 1406 and a graphics adapter 1412 are coupled to the memory controller hub 1420, and a display 1418 is coupled to the graphics adapter 1412. A storage device 1408, keyboard 1410, pointing device 1414, and network adapter 1416 are coupled to the I/O controller hub 1422. Other embodiments of the computer 1400 have different architectures.

In the embodiment shown in FIG. 14, the storage device 1408 is a non-transitory computer-readable storage medium such as a hard drive, compact disk read-only memory (CD-ROM), DVD, or a solid-state memory device. The memory 1406 holds instructions and data used by the processor 1402. The pointing device 1414 is a mouse, track ball, touchscreen, or other type of pointing device, and may be used in combination with the keyboard 1410 (which may be an on-screen keyboard) to input data into the computer system 1400. The graphics adapter 1412 displays images and other information on the display 1418. The network adapter 1416 couples the computer system 1400 to one or more computer networks, such as network 150.

The types of computers used by the entities of FIG. 1 can vary depending upon the embodiment and the processing power required by the entity. For example, the monitoring module 185 might include multiple blade servers working together to provide the functionality described while a client device 130 might be a tablet or laptop. Furthermore, the computers can lack some of the components described above, such as keyboards 1410, graphics adapters 1412, and displays 1418.

### ADDITIONAL CONSIDERATIONS

Some portions of above description describe the embodiments in terms of algorithmic processes or operations. These algorithmic descriptions and representations are commonly used by those skilled in the computing arts to convey the substance of their work effectively to others skilled in the art. These operations, while described functionally, computationally, or logically, are understood to be implemented by computer programs comprising instructions for execution by a processor or equivalent electrical circuits, microcode, or the like. Furthermore, it has also proven convenient at times, to refer to these arrangements of functional operations as modules, without loss of generality.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate +/−10% unless another meaning is apparent from the context. From example, “approximately ten” should be understood to mean “in a range from nine to eleven.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs for a system and a process for automatically classifying subvisible particles. Thus, while particular embodiments and applications have been illustrated and described, it is to be understood that the described subject matter is not limited to the precise construction and components disclosed. The scope of protection should be limited only by any claims that may ultimately issue.

What is claimed is:

1. A computer-implemented method for management of pooled medicinal product across studies, the method comprising:
pooling medicinal product across a plurality of clinical trial studies into one or more pools based on characteristics of the clinical trial studies;
allocating one or more batches of medicinal product to each pool of studies;
for each pool of studies, assigning a unique identifier to each medicinal product in the one or more batches of medicinal product allocated to the pool of studies;
receiving, from a treatment-site client device, an order request for a quantity of medicinal product;
identifying a first pool of studies associated with the treatment-site client device;
generating a fulfillment list of medicinal product selected from the one or more batches of medicinal product allocated to the first pool of studies; and
providing the fulfillment list for filling the order request.

2. The computer-implemented method of claim 1, wherein pooling the medicinal product across the plurality of clinical trial studies comprises:
identifying, for each clinical trial study, a set of one or more countries for conducting the clinical trial study;
identifying, for each country, protocol labeling parameters; and
pooling the medicinal product based on overlapping protocol labeling parameters between countries.

3. The computer-implemented method of claim 1, wherein pooling the medicinal product across the plurality of clinical trial studies comprises:
identifying, for each clinical trial study, blinding parameters; and
pooling the medicinal product based on the blinding parameters for the clinical trial studies.

4. The computer-implemented method of claim 1, further comprising:
applying a model to forecast demand for each pool of studies based on the clinical trial studies in the pool of studies,
wherein allocating the batches of medicinal product across the pools of studies is based on the forecasted demands for the pools of studies.

5. The computer-implemented method of claim 1, further comprising:
applying a model to forecast demand for a treatment site associated with the treatment-site client device based on participants enrolled at the treatment site;
wherein generating the fulfillment list of medicinal product comprises allocating an amount of medicinal product based on the forecasted demand for the treatment site.

6. The computer-implemented method of claim 1, wherein, for each pool of studies, assigning the unique identifier to each medicinal product allocated to the pool of studies comprises random assignment of double permutated alphanumeric identifiers.

7. The computer-implemented method of claim 1, further comprising:
storing the unique identifiers for the medicinal product for each pool of studies in a database associated with one or more protocols for the clinical trial studies in the pool of studies.

8. The computer-implemented method of claim 7, further comprising:
receiving, via the treatment-site client device, an identifier for a participant and a first unique identifier for the medicinal product associated with the first pool of studies;
identifying that the participant is enrolled in a first study in the first pool of studies;
accessing the protocol associated with the first study from the database; and
transmitting, to the treatment-site client device, the protocol associated with the first study.

9. The computer-implemented method of claim 1, further comprising:
accessing a label image for inclusion on the medicinal product allocated to the first pool of studies; and
encoding the label image for presentation on digital display labels affixed to the medicinal product allocated to the first pool of studies.

10. The computer-implemented method of claim 9, further comprising:
identifying protocol labeling parameters associated with a first country where the treatment site is located,
wherein accessing the label image is based on the protocol labeling parameters associated with the first country.

11. A non-transitory computer-readable storage medium storing instructions for management of pooled medicinal product across studies, the instructions that, when executed by a computer processor, cause the computer processor to perform operations comprising:
pooling medicinal product across a plurality of clinical trial studies into one or more pools based on characteristics of the clinical trial studies;
allocating one or more batches of medicinal product to each pool of studies;
for each pool of studies, assigning a unique identifier to each medicinal product in the one or more batches of medicinal product allocated to the pool of studies;
receiving, from a treatment-site client device, an order request for a quantity of medicinal product;
identifying a first pool of studies associated with the treatment-site client device;
generating a fulfillment list of medicinal product selected from the one or more batches of medicinal product allocated to the first pool of studies; and
providing the fulfillment list for filling the order request.

12. The non-transitory computer-readable storage medium of claim 11, wherein pooling the medicinal product across the plurality of clinical trial studies comprises:
identifying, for each clinical trial study, a set of one or more countries for conducting the clinical trial study;
identifying, for each country, protocol labeling parameters; and
pooling the medicinal product based on overlapping protocol labeling parameters between countries.

13. The non-transitory computer-readable storage medium of claim 11, wherein pooling the medicinal product across the plurality of clinical trial studies comprises:
identifying, for each clinical trial study, blinding parameters; and
pooling the medicinal product based on the blinding parameters for the clinical trial studies.

14. The non-transitory computer-readable storage medium of claim 11, the operations further comprising:
applying a model to forecast demand for each pool of studies based on the clinical trial studies in the pool of studies,
wherein allocating the batches of medicinal product across the pools of studies is based on the forecasted demands for the pools of studies.

15. The non-transitory computer-readable storage medium of claim 11, the operations further comprising:
applying a model to forecast demand for a treatment site associated with the treatment-site client device based on participants enrolled at the treatment site;
wherein generating the fulfillment list of medicinal product comprises allocating an amount of medicinal product based on the forecasted demand for the treatment site.

16. The non-transitory computer-readable storage medium of claim 11, wherein, for each pool of studies, assigning the unique identifier to each medicinal product allocated to the pool of studies comprises random assignment of double permutated alphanumeric identifiers.

17. The non-transitory computer-readable storage medium of claim 11, the operations further comprising:
storing the unique identifiers for the medicinal product for each pool of studies in a database associated with one or more protocols for the clinical trial studies in the pool of studies.

18. The non-transitory computer-readable storage medium of claim 17, the operations further comprising:
receiving, via the treatment-site client device, an identifier for a participant and a first unique identifier for the medicinal product associated with the first pool of studies;
identifying that the participant is enrolled in a first study in the first pool of studies;
accessing the protocol associated with the first study from the database; and
transmitting, to the treatment-site client device, the protocol associated with the first study.

19. The non-transitory computer-readable storage medium of claim 11, the operations further comprising:
accessing a label image for inclusion on the medicinal product allocated to the first pool of studies; and
encoding the label image for presentation on digital display labels affixed to the medicinal product allocated to the first pool of studies.

20. The non-transitory computer-readable storage medium of claim 19, the operations further comprising:
identifying protocol labeling parameters associated with a first country where the treatment site is located,
wherein accessing the label image is based on the protocol labeling parameters associated with the first country.
